SEQUENCE LISTING (4 sequences)

SEQ ID NO: 1 - DNA, 21 bp, artificial
  synthetic nuceotide produced to interfere with mRNA of target
ggactcgctttctcaggtttc

SEQ ID NO: 2 - DNA, 21 bp, artificial
  synthetic nuceotide produced to interfere with mRNA of target
ccattctgaatcggtaagcga

SEQ ID NO: 3 - DNA, 21 bp, artificial
  synthetic nuceotide produced to interfere with mRNA of target
ccagagccgtaagttcatcat

SEQ ID NO: 4 - DNA, 21 bp, artificial
  synthetic nuceotide produced to interfere with mRNA of target
gaacctgtatgcaaggcctgg

(19) United States
(12) Patent Application Publication
(10) Pub. No.: US 2009/0209619 A1
(43) Pub. Date: Aug. 20, 2009

(54) Depression of herg k+ channel function in mammallan cells and applications to the control of cancer cells division

(76) Inventors: Zhiguo Wang, Montreal (CA); Baofeng Yang, Heilongjiang (CN)

Correspondence Address: Ralph A. Dowell of DOWELL & DOWELL P.C., 2111 Eisenhower Ave, Suite 406, Alexandria, VA, 22314, US

(21) Appl. No.: 11/665,368

(22) PCT Filed: Oct. 14, 2005
(86) PCT No.: PCT/CA2005/001574

§ 371 Date: Apr. 13, 2007

Related U.S. Application Data
(60) Provisional application No. 60/618,142, filed on Oct. 14, 2004.

(51) Int. Cl. A61K 31/7088; A61K 31/18; A61K 31/445; A61K 48/00

(52) U.S. Cl. 514/44 A; 514/605; 514/327

(57) ABSTRACT

The use of an HERG channel inhibitor for controlling the proliferation of cancer cells. Examples of such HERG channel inhibitors include dofetilide, cisapride, E-4031 and a siRNA molecule targeting a sequence involved in the expression of an HERG channel. Other ERG channels are also targets for these inhibitors.

This application claims priority from U.S. Provisional Patent Applications Ser. No. 60/618,142 filed Oct. 14, 2004.

### FIELD OF THE INVENTION

The present invention relates to the control of cell division. More specifically, the present invention is concerned with the depression of HERG K⁺ channel function in mammalian cells and applications to the control of cancer cells division.

### BACKGROUND OF THE INVENTION

Cancer is a major cause of mortality in the developed world. There is no effective treatment for many types of cancers. In addition, for many cancers for which such a treatment exists, the treatment produces undesirable side effects. This is in part due to the fact that such treatments often target dividing cells. Indeed, since cancer involves rapidly dividing cells, such treatments seem appropriate. However, these treatments may affect any tissue in which cells are dividing.

Among all cancers, breast cancer is one of the most prevalent cancer. It is a leading cause of cancer death for women worldwide. The current methods of treatment in use for this cancer are surgery, radiation, chemotherapy, hormone therapy, and biological therapy. However, these treatments are only efficient in some patients.

While details have been given hereinabove about breast cancer, similar problems occur in many other types of cancer.

Against this background, there exists a need in the industry to provide novel methods and compounds for the control of mammalian cells division, for example to control cancer cells division.

The present description refers to a number of documents, the content of which is herein incorporated by reference in their entirety.

### OBJECTS OF THE INVENTION

An object of the present invention is therefore to provide novel compounds having an affinity for the lungs.

### SUMMARY OF THE INVENTION

In a broad aspect provides, the invention provides the use of an HERG channel inhibitor for controlling the proliferation of cancer cells.

In another broad aspect, the invention provides the of an ERG channel inhibitor for the manufacture of a pharmaceutical composition of matter for controlling the proliferation of mammalian cancer cells.

In yet other broad aspects, the invention provides the use of a HERG channel inhibitor for controlling the proliferation of cancer cells and a method for reducing tumor growth, the method comprising the administration of a therapeutically effective amount of a ERG channel inhibitor to a subject.

In yet other broad aspects, the invention provides a method for lowering the progression of cancerous cell proliferation, the method comprising administering to a subject in need thereof a therapeutically effective amount of a ERG channel inhibitor, and a method for treating cancer in a human, the method comprising the administration of a therapeutically effective amount of a HERG channel inhibitor.

In an other broad aspect, the invention provides a method of inhibiting a HERG channel in a cancer patient, comprising administering an effective amount of an siRNA molecule and such an siRNA molecule.

In another broad aspect, the invention provides a method of inhibiting a HERG channel in a cancer patient, comprising administering an effective amount of an HERG channel inhibitor selected from dofetilide, E4031, and cisapride to a patient in need thereof. Alternatively, an effective amount of an anti-arrhythmic agent or an esophageal sphincter contracting agent is administered to the.

Non-limiting examples of a cancer treatable with the present invention includes breast cancer, neuroblastoma and atrial cancer, among others.

Advantageously, the tested ERG inhibitors have shown a relatively large effect on cancer cell division. Since some of the ERG inhibitors tested, namely cisapride and dofetilide, are substances currently used for the treatment of other conditions, it can be expected that the claimed treatments could be used for cancer treatment without causing excessive side effects.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawings:

FIG. 1 illustrates the inhibition of proliferation by HERG K+ channel blockers dofetilide (1 mM), E4031 (1 mM) or cisapride (1 mM) in various tumor cell lines: (A) Comparison of population doubling time (PDT) showing that HERG blockers prolonged PTD in all cell lines tested, indicating slowing of cell proliferation; (B) Percent changes of PTD in presence of drugs over control;

FIG. 2 illustrates the inhibition of proliferation by siRNAs targeting HERG K+ channel mRNA in various tumor cell lines: (A) Comparison of population doubling time (PDT); (B) Percent changes of PTD in presence of siRNA over control;

FIG. 3 illustrates the inhibition of tumor growth by HERG K+ channel blockers dofetilide (5 mg/tumor), E4031 (5 mg/tumor) or cisapride (5 mg/tumor) in nude mice inoculated subcutaneously with HERG-expressing breast cancer cell lines MCF-7 (A) or SK-BR-3 (B); tumor growth was determined by changes of tumor volume between the onset of drug application to the tumor (day 1) and day 7; panel (C) shows the percent decreases in tumor volume over control on day 7; and

FIG. 4 illustrates the inhibition of tumor growth by siRNA-1 targeting HERG K+ channel mRNA in nude mice inoculated subcutaneously with HERG-expressing breast cancer cell lines MCF-7 (A) or SK-BR-3 (B); tumor growth was determined by changes of tumor volume between the onset of drug application to the tumor (day 1) and day 7; panel (C) shows the percent changes of tumor volume over control on day 7.

### DETAILED DESCRIPTION

K+Channels and Cancers

Although it is still too early to catalogue cancer as a channelopathy (a disorder arising directly from ion channel dysfunction) there is mounting evidence pointing to the involvement of ion channels in cancer progression and pathology [1]. The contribution of ion channels to the neoplastic phenotype includes the control of cell proliferation and apoptosis, as well as the regulation of invasive growth and metastasis. The presence of K⁺ channels in tumor cells has been confirmed in published studies. The contributions of ion channels to the neoplastic phenotype are as diverse as the ion channel families in tumor cells. There is evidence that K+ channel activity is required for G1 progression of cell cycle in different cell backgrounds, suggesting that K+ channel activity is involved in early-stage cell proliferation [2-12].

Ion channels can affect cell proliferation in several ways. For example, through an oscillation of the transmembrane potential. In general, cancer cells possess more positive transmembrane potentials relative to healthy cells of the same histological origin. The membrane depolarization has been believed to be involved in unlimited tumor cell proliferation, presumably due to facilitation of Ca²⁺ entry through activation of voltage-dependent Ca²⁺ channels at less negative voltages. K⁺ channels are known to be a involved in the production of cell membrane potential, and are thereby a regulator of cell proliferation.

For example, Marino et al [7] investigated the electrical potentials in 110 women with palpable breast masses. The tumor site was found to be significantly electropositive compared with control sites only when the tumor was a cancer, as determined by a subsequent biopsy. Similarly, the resting membrane potentials of unsynchronized MCF-7 cells during exponential growth phase oscillates from −58.6 mV to −2.7 mV. As a second mechanism, Ca²⁺ entry through Ca²⁺ channels and subsequent intracellular Ca²⁺ mobilization favor tumor cell growth [13-15]. Activation of K⁺ channels hyperpolarizes membrane so as to increase the driving force (electrochemical gradient) for Ca²⁺ influx, thereby interacting with Ca²⁺-dependent cell cycle control proteins [16]. This mechanism was proposed by Nilius and Wohlrab [17] to explain their observation that K+channel blockage inhibited proliferation of melanoma, T-lymphocytes and human breast carcinomas. Third, K⁺ channels regulate cell volume; opening of K⁺ channels carries K⁺ efflux leading to cell shrinkage. Rouzaire-Dubois & Dubois demonstrated that K⁺ channel blockers increased the cell volume and decreased the rate of cell proliferation; proliferation was fully inhibited when cell volume was increased by 25% [18-19]. Finally, intracellular growth-promoting factors have been implicated in the regulation of tumor cell growth by K⁺ channels. Xu et al [20] demonstrated that in human myeloblastic leukemia ML-1 cells suppression of K⁺channels prevented the activation of extracellular signal-regulated protein kinase 2 in response to endothelium growth factor and serum.

It has recently been demonstrated that HERG (the human ether-a-go-go-related gene) expression facilitates tumor cell proliferation caused by tumor necrosis factor-alpha (TNF-α), and HERG and TNF receptor 1 co-localize on the cytoplasmic membrane, which is well correlated with greater activities of the nuclear transcription factor-κB, NFκB, in HERG-expressing tumor cells than in tumor cells that do not express HERG [21].

HERG K⁺ Channel and Breast Cancers

Several K⁺ channels have been implicated in breast cancer cell proliferation. Minoxidil, an activator of ATP-sensitive K+ channel, was found to stimulate growth of MCF-7 human breast cancer cells [22]. K⁺ channel blockers inhibit breast cancer cell proliferation. For instance, dequalinium and amiodarone had inhibitory effects on MCF-7 proliferation and potentiated the growth-inhibitory effects of tamoxifen on MCF-7 and MDA-MB-231 [22]. One study provided evidence for Kv1.3 underlying MCF-7 cell growth by investigating Kv1.3 expression in 60 human breast cancer specimens with immunohistochemistry [22]; however, the cause-effect relationship was not established in this study. Another study also in MCF-7 cells suggests the role of a-DTX-sensitive Kv1.1 in proliferation because partial blockade of the channels by a-DTX reduced proliferation by 30% [23]. A K⁺ channel gene called ether-a-go-go (EAG) was cloned from human breast carcinoma MCF-7 cells and noticeably, EAG mRNA was not detectable in normal human breast. Expression of EAG was also found in several other breast tumor cell lines including COLO-824, EFM-19, BT474 cells [25-26]. Extracellular perfusion of astemizole inhibited EAG current by ˜20% and cell proliferation by ˜23%. Moreover, the EAG mRNA expression was modulated during the cell cycle [27].

It has been found that HERG K⁺ channel expression facilitates the tumor cell proliferation caused by TNF-α at concentrations <1 ng/ml [21]. The effect was observed only in HERG-expressing cells such as the breast cancer cell SK-BR-3, but not in the tumor cells without endogenous HERG (A549 and SK-Mel-28 cells). As to be described below, the role of HERG in promoting proliferation has also been recently verified in several other breast cancer cell lines. Noticeably, a group of medications called selective estrogen receptor modulators (SERMs)—for example, tamoxifen, used for treating breast cancers, have been found to potentially inhibit HERG K⁺ channels [28-29].

The extensive use of long-term adjuvant tamoxifen has resulted in saving the lives of 400,000 women with breast cancer [30]. ERG expression has been found in a variety of tumor cell lines of different histogenesis but absent from the healthy cells from which the respective tumor cells are derived. Correspondingly, HERG function (channel conductance) is also enhanced in transformed cells compared to healthy cells of the same histological origins.

Inhibition of Cancer Cell Proliferation

Methods

The human breast cancer cell lines MCF-7, BT132, and Sk-Br-3, human neuroblastoma cell line SHSY5Y, murine atrial tumor cell line HL-1 were used. These cell are known to express HERG channels. For chemical blocking of HERG conductance, tumor cells were incubated with dofetilide (1 μM), the anti-arrhythmic agent E4031 (1 μM) or cisapride (1 μM) for 30 min in serum-free medium. Control experiments wherein no dofetilide, no E-4031 and no cisapride was used were also performed.

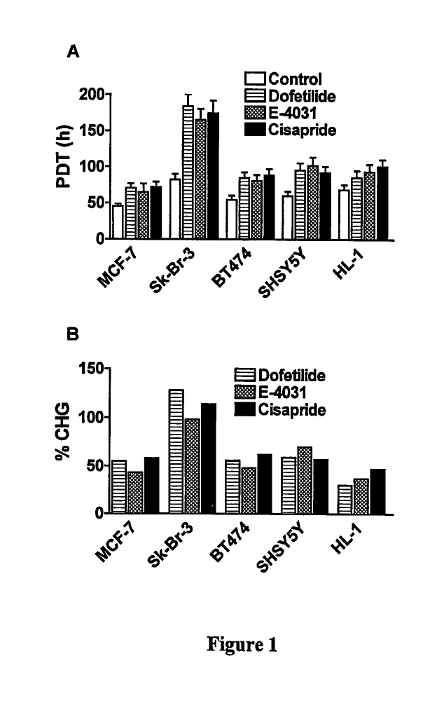

Cell proliferation was assessed by characterizing the log phase growth with population doubling time (PDT) calculated by the equation: 1/(3.32*(log N_{H}−log N_{I})/(t₂−t₁)), where N_{H} is the number of cells harvested at the end of the growth period (at time t₂) and N_{I} is the number of cells at 5 hours (t₁) after seed. Cells were counted by a flow cytometer (EPICS XL; Beckman Coulter Canada, Inc.), and the number obtained at 5 h (t₁) after seeding was taken as an initial cell number (N_{I}), and the number at 48 h (t₂) after seeding was taken as an endpoint number (N_{H}). The longer the PDT is, the slower the growth is. The results of these experiments are shown in Table 1 and in FIG. 1.

TABLE 1
Effects of HERG channel blockers on cancer cell proliferation determined
by population doubling time in hours and % changes over control.

| | MCF-7 | Sk-Br-3 | BT132 | SHSY5Y | HL-1 |
|---|---|---|---|---|---|
| Control | 45.4 ± 3.2 | 82.3 ± 7.6 | 54.4 ± 5.6 | 59.8 ± 6.2 | 68.2 ± 7.1 |
| | (n = 5) | (n = 6) | (n = 4) | (n = 5) | (n = 4) |
| Dofetilide | 70.4 ± 6.2* | 184.0 ± 16.6* | 84.8 ± 7.5* | 95.2 ± 10.2* | 85.5 ± 9.2 |
| (1 μM) | 55% | 128% | 56% | 59% | 30% |
| E-4031 | 64.9 ± 11.5* | 165.3 ± 15.2* | 80.5 ± 8.2* | 101.8 ± 11.5* | 93.5 ± 10.0* |
| (1 μM) | 43% | 98% | 48% | 70% | 37% |
| Cisapride | 71.7 ± 7.3* | 174.3 ± 17.7* | 88.1 ± 9.0* | 92.0 ± 8.3* | 100.3 ± 9.8* |
| (1 μM) | 58% | 114% | 62% | 57% | 47% |

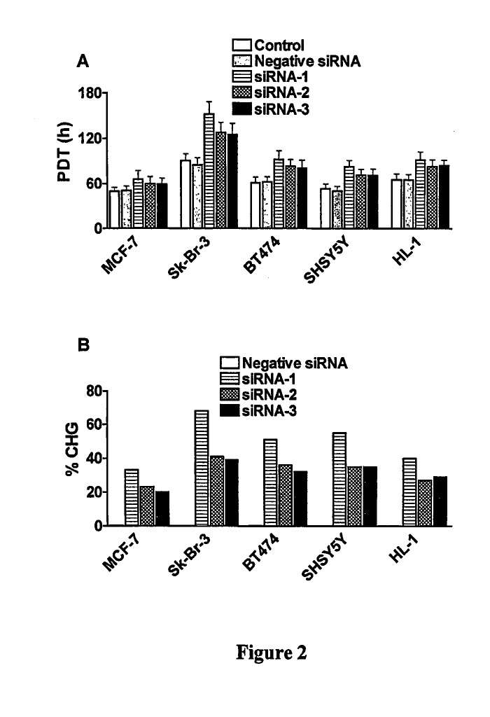

In experiments involving interference RNA, in this case siRNA, the cells were transfected with siRNA using lipofectamine-2000 as a carrier to deliver the siRNA to the cells, according to the manufacturer's protocols. The sequence of our siRNA targeting HERG position 3498 bp is GGACTCGCTTTCTCAGGTTTC (SEQ ID NO:1). A negative control targeting the following sequence: CCATTCTGAATCGGTAAGCGA (SEQ ID NO:2) has been used. The siRNA is designed as cassette using U6 as the promoter and its efficacy was validated by its ability to decrease HERG mRNA by around 78% determined by real-time RT-PCR. The results of these experiments are shown in Table 2 and in FIG. 2. SiRNA targeting the following 2 target sequences: CCAGAGCCGTAAGTTCATCAT (SEQ ID 03) (starting at 255 bp) and GAACCTGTATGCAAGGCCTGG (SEQ ID 04) (starting at 2609 bp) has also been tested. As an siRNA cassette is used, these sequences (SEQ ID 01-04) are also DNA sequences that are injected in the cells to produce in situ the siRNA that will interfere with expression of the HERG gene.

TABLE 2
Effects of siRNA targeting HERG mRNA on cancer cell proliferation determined
by population doubling time in hours and % changes over control.

| | MCF-7 | Sk-Br-3 | BT132 | SHSY5Y | HL-1 |
|---|---|---|---|---|---|
| Control | 49.4 ± 5.1 | 90.5 ± 8.9 | 61.3 ± 7.6 | 53.0 ± 6.6 | 65.6 ± 7.6 |
| | (n = 4) | (n = 4) | (n = 3) | (n = 3) | (n = 3) |
| Negative | 50.7 ± 5.9 | 84.8 ± 9.6 | 62.5 ± 6.7 | 50.4 ± 5.7 | 65.5 ± 7.2 |
| Control | 0.3% | −3% | 0.2% | −5% | 0% |
| siRNA | | | | | |
| siRNA | 65.7 ± 11.5* | 152.1 ± 16.2* | 92.5 ± 11.2* | 82.8 ± 8.1* | 91.8 ± 10.8* |
| targeting | 33% | 68% | 51% | 55% | 40% |
| SEQ ID 01 | | | | | |
| siRNA | 60.1 ± 9.2* | 127.6 ± 13.5* | 83.4 ± 9.0* | 71.6 ± 7.8* | 83.2 ± 8.9* |
| targeting | 23% | 41% | 36% | 35% | 27% |
| SEQ ID 03 | | | | | |
| siRNA | 59.3 ± 8.1* | 124.9 ± 14.7* | 80.9 ± 10.6* | 71.3 ± 8.4* | 84.5 ± 7.3* |
| targeting | 20% | 39% | 32s% | 35% | 29% |
| SEQ ID 04 | | | | | |

Results and Discussion

In table 1, the number of independent experiments is indicated by the values in parenthesis in the line regarding the control experiments. *p<0.05 vs Control. Table 1 summarizes the PDT results and shows the influence of dofetilide, E-4031, and cisapride on cell proliferation in the tested cell lines.

As seen from Table 1, dofetilide, E4031, and cisapride have statistically significantly increased the PDT in the human breast cancer cell lines MCF-7, BT132, and Sk-Br-3, the human neuroblastoma cell line SHSY5Y, and in murine atrial tumor cell line HL-1. Therefore, blockade of HERG conductance by dofetilide, E-4031 or cisapride inhibits proliferations of various cancer cells.

Table 2 illustrates the influence of the siRNA targeting SEQ ID 01, 03 and 04 on cell growth by indicating the PDT in controls, cells transfected with the siRNA targeting the sequence SEQ ID 01, 03 and 04 and cells transfected with the siRNA targeting the sequence SEQ ID 2. Results are shown for different cell lines. The number of independent experiments is indicated by the values in between parentheses in the control line. *p<0.05 vs Control.

As seen from Table 2, depression of HERG function by siRNA that specifically knocks down the HERG gene inhibits proliferations of various cancer cells. HERG K+ channel is therefore a target for chemotherapy and gene therapy of cancers.

Inhibition of Tumor Growth

Methods

Female athymic nu/nu mice (6-8 weeks old) were housed five/cage in a pathogen-free environment under controlled conditions of light and humidity in the Animal House of Harbin Medical University on a standard sterilizable laboratory diet. Mice were quarantined 1 week before experimental manipulation; at the end of the quarantine mice were inoculated subcutaneously with HERG-expressing breast cancer cell lines MCF-7 and SK-BR-3 (2×10⁶) in 0.1 ml of Matrigel. Tumor size was measured weekly using calipers and the histological appearance, grading, angiogenesis evaluated by histology. The volume of the tumor was calculated using the formula: 4πr₁²r₂/3 (r1; short axis; r2; long axis) and converted into natural logarithms.

After the tumor volume had achieved 80 mm³, tumor bearing mice for each inoculum were randomized into control and drug groups (6 mice/group): three drug groups of animals were treated with HERG inhibitors (dofetilide, E-4031 or cisapride) at the same dosage (5 μg/tumor).

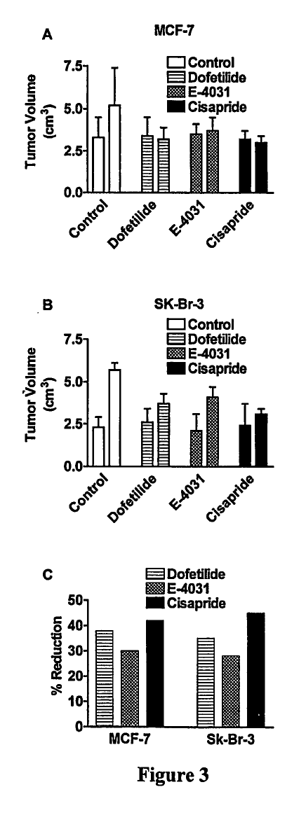

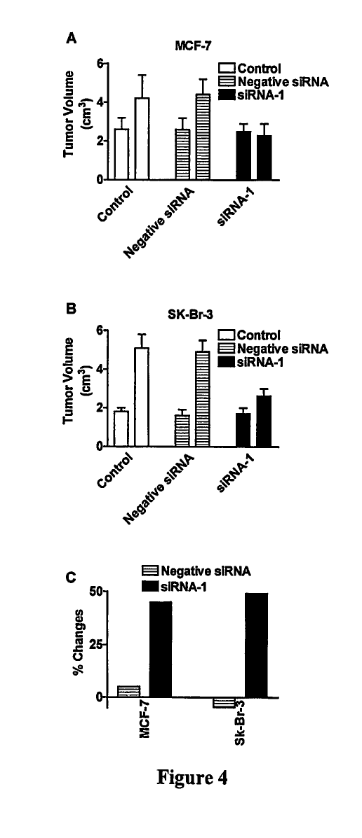

The drugs were injected directly in a single dose into the tumor mass. For siRNA experiments, siRNA targeting the SEQ ID 01 (1 μg/Tumor) or the negative control siRNA (SEQ ID 2) (1 μg/Tumor) were treated with lipofectamine 2000 before being injected into the tumor mass. Comparison of tumor size between day 1 (the day when the mice were treated with drugs or siRNA) and day 7 (7 days after drug treatment) was made. Results are found in Tables 3 and 4 and in FIGS. 3 and 4.

Results

Table 3 illustrates the tumor volumes in mice injected with dofetilide, E-4031 and cisapride on the day the mice were injected with the drug (day1) and 7 days after the drug treatment (day 7). Table 3 also mentions the percentage of change in tumor volume in drug treated tumors as compared to controls. The number of animals studied is indicated by the values between parentheses in the control group line. Statistical significance is indicated by *: p<0.05 vs Day 1; +: p<0.05 vs. Control.

TABLE 3
Effects of HERG channel blockers on tumor growth in nude mice,
determined by tumor volume (cm³) and % changes over control.

| | MCF-7 | Sk-Br-3 |
|---|---|---|
| Control | | |
| Day 1 | 3.3 ± 1.2 | 2.3 ± 0.6 |
| Day 7 | 5.2 ± 2.2* | 5.7 ± 0.4* |
| | (n = 6) | (n = 6) |
| Dofetilide (5 μg/Tumor) | | |
| Day 1 | 3.4 ± 1.1 | 2.6 ± 0.8 |
| Day 7 | 3.2 ± 0.7⁺ | 3.7 ± 0.6⁺ |
| | −38% | −35% |
| E-4031 (5 μg/Tumor) | | |
| Day 1 | 3.5 ± 0.6 | 2.1 ± 1.0 |
| Day 7 | 3.7 ± 0.8⁺ | 4.1 ± 0.6*⁺ |
| | −30% | −28% |
| Cisapride (5 μg/Tumor) | | |
| Day 1 | 3.2 ± 0.5 | 2.4 ± 1.3 |
| Day 7 | 3.0 ± 0.4⁺ | 3.1 ± 0.3⁺ |
| | −42% | −45% |

A seen, tumor growth between day 1 and day 7 was statistically significant in both tumor types for the control group. Dofetilide, E-4031 and cisapride all reduced significantly tumor growth in both tumor types as compared to the control. In many cases, there was no significant growth between day 1 and day 7 in drug-treated cells.

Table 4 illustrates the tumor volumes in mice injected with THE siRNA having SED ID 01, the siRNA targeting SEQ ID 02 and control mice on the day the mice were injected with the drug (day1) and 7 days after the drug treatment (day 7). Table 4 also mentions the percentage of change in tumor volume in drug treated tumors as compared to controls. The number of animals studied is indicated by the values between parentheses in the control group line. Statistical significance is indicated by *: p<0.05 vs Day 1; +: p<0.05 vs. Control.

TABLE 4
Effects of siRNA targeting HERG mRNA on tumor growth in nude
mice, determined by tumor volume and % changes over control.

| | MCF-7 | Sk-Br-3 |
|---|---|---|
| Control | | |
| Day 1 | 2.6 ± 0.6 | 1.8 ± 0.2 |
| Day 7 | 4.2 ± 1.2* | 5.1 ± 0.7* |
| | (n = 6) | (n = 6) |
| Negative siRNA (1 μg/Tumor) | | |
| Day 1 | 2.6 ± 0.6 | 1.6 ± 0.3 |
| Day 7 | 4.4 ± 0.8* | 4.9 ± 0.6* |
| | 5% | −4% |
| siRNA (1 μg/Tumor) | | |
| Day 1 | 2.5 ± 0.4 | 1.7 ± 0.3 |
| Day 7 | 2.3 ± 0.6⁺ | 2.6 ± 0.4⁺ |
| | −45% | −49% |

A seen, tumor growth between day 1 and day 7 was statistically significant in both tumor types for the control group and for the group treated with the negative siRNA. The siRNA targeting the SEQ ID 01 reduced significantly tumor growth in both tumor types as compared to the control.

The above results suggest the use of an HERG channel inhibitor for controlling the proliferation of cancer cells.

The above results further suggest the use of an ERG, such as for example and non-limitingly, the use of an HERG channel inhibitor for the manufacture of a pharmaceutical composition of matter for controlling the proliferation of mammalian cancer cells. The pharmaceutical composition of matter may for controlling the proliferation of cancer cells in a non-human mammal or in a human. Non-limiting examples of suitable HERG channel inhibitors include dofetilide, the anti-arrhythmic agent E-4031, and cisapride.

For example, the HERG channel inhibitor may be administered intra-tumorally. However, other routes of administration, such as for example administration through inhalotherapy or trough an oral medication are also within the scope of the invention. Other non-limiting examples of other routes of administration include intravenous administration, parenteral administration, oral administration trough capsules, oral administration trough tablets and oral administration trough a liquid solution.

The HERG channel inhibitor is administered in an amount of from about 1 ng to about 1 g, and in some embodiments of the invention, in an amount of from about 1 μg to about 1 mg. In other embodiments of the invention, the HERG channel inhibitor is administered in an amount of from about 0.1 ng/(tumor cm³) to about 1 g/(tumor cm³) or in an amount of from about 0.1 μg/(tumor cm³) to about 10 μg/(tumor cm³).

In other examples of implementation, the HERG channel inhibitor is an interference RNA that downregulates the expression of the HERG channel. The interference RNA may includes an siRNA including a sense strand comprising a portion whose target sequence is at least 90% identical to a sequence listed in any of SEQ ID NOs: 1, 3 and 4 over at least 15 continuous nucleotides. For example, the interference RNA includes an siRNA including a sense strand comprising a portion whose target sequence 100% identical to a sequence listed in any of SEQ ID NOs: 1, 3 and 4. The siRNA mat be dissolved in a pharmaceutically acceptable carrier.

The above results also suggest a method for reducing tumor growth, the method comprising the administration of a therapeutically effective amount of an ERG channel inhibitor to a subject, and a method for treating cancer in a human, the method comprising the administration of a therapeutically effective amount of a HERG channel inhibitor.

Furthermore, the above results show examples of an RNAi agent targeted to a target transcript that encodes a protein involved in development, pathogenesis, or symptoms of an HERG-related disease, such as for example cancer. The transcript may encode at least a portion of an HERG channel.

While the above animal model studies were performed with a drug injected in a single dose, it is within the scope of the present invention to administer a suitable drug in any other suitable manner. For example, the drug may be administered in many dosages spaced by regular or irregular time intervals.

In some examples of implementation, the RNAi agent is a siRNA, that may include duplex portion of between about 15 and about 25 nucleotides long. In other examples of implementation, the siRNA has a polynucleotide sequence having at least one strand that is substantially complementary to at least ten but no more than thirty consecutive nucleotides coding for at least a portion of an HERG channel and that reduces the expression of HERG nucleic acid or protein. The siRNA may be double-stranded.

Dofetilide and E-4031 are known to be anti-arrhythmic agents. Also, cisapride is a known esophageal sphincter contracting agent. Therefore, these results suggest that other anti-arrhythmic agents and esophageal sphincter contracting agents have a potential to cause similar effects on cell proliferation of cancerous cells.

All references cited and/or discussed in this specification are incorporated herein by reference in their entirety and to the same extent as if each reference was individually incorporated by reference.

The in vivo experiments in mice and in vitro experiments in various cancer cell lines described in the specification may be predictive of biological effects in humans or other mammals and/or may serve as animal models for use of the present invention in humans or other mammals for the treatment of cancers involving cells similar to the cell lines that were used, for the treatment of cancer types similar to the cancer types that were investigated in these experiments, and for the treatment of other types of cancer. These experiments may also be predictive of regulatory effects on tumor growth and regression.

Although the present invention has been described hereinabove by way of preferred embodiments thereof, it can be modified, without departing from the spirit and nature of the subject invention as defined in the appended claims.

### REFERENCES

1. Wang Z. Roles of K+ channels in regulating tumor cell proliferation and apoptosis. Pflügers Arch 2004; 448:274-286.
2. Redmann K, Muller V, Tanneberger S, Kalkoff W. The membrane potential of primary ovarian tumor cells in vitro and its dependence on the cell cycle. Acta Biol Med Ger 1972; 28:853-856.
3. Smith T C, Levinson C. Direct measurement of the membrane potential of Ehrlich ascites tumor cells: lack of effect of valinomycin and ouabain. J Membr Biol 1975; 23:349-365.
4. Lymangrover J. Pearlmutter A F, Franco-Saenz R, Saffran M. Transmembrane potentials and steroidogenesis in normal and neoplastic human adrenocortical tissue. J Clin Endocrinol Metab 1975; 41:697-706.
5. Binggeli R, Cameron I L. Cellular potentials of normal and cancerous fibroblasts and hepatocytes. Cancer Res 1980; 40:1830-1835.
6. Stevenson D, Binggeli R, Weinstein R C, Keck J G, Lai M C, Tong M J. Relationship between cell membrane potential and natural killer cell cytolysis in human hepatocellular carcinoma cells. Cancer Res 1989; 49:48424845.
7. Marino A A, Morris D M, Schwalke M A, Iliev I G, Rogers S. Electrical potential measurements in human breast cancer and benign lesions Tumor Biol 1994; 15:147-152.
8. Wonderlin W F, Woodfork K A, Strobl J S. Changes in membrane potential during the progression of MCF-7 human mammary tumor cells through the cell cycle. J Cell Physiol 1995; 165:177-185.
9. Zhang J, Davidson R M, Wei M D, Loew L M. Membrane electric properties by combined patch clamp and fluorescence ratio imaging in single neurons. Biophys J 1998; 74:48-53.
10. Pandiella A, Magni M, Lovisolo D, Meldolesi J. The effect of epidermal growth factor on membrane potential. Rapid hyperpolarization followed by persistent fluctuations. J Biol Chem 1989; 264:12914-12921.
11. Lang F, Friedrich F, Kahn E, Woll E, Hammerer M, Waldegger S, Maly K, Grunicke H. Bradykinin-induced oscillations of cell membrane potential in cells expressing the Ha-ras oncogene. J Biol Chem 1991; 266:4938-4942.
12. Lang F, Waldegger S, Woell E, Ritter M, Maly K, Grunicke H. Effects of inhibitors and ion substitutions on oscillations of cell membrane potential in cells expressing the RAS oncogene. Pflügers Arch 1992; 421:416-24.
13. Lee Y S, Sayeed M M, Wurster R D. Inhibition of cell growth and intracellular Ca2+ mobilization in human brain tumor cells by Ca2+ channel antagonists. Mol Chem Neuropathol 1994; 22:81-95.
14. Kim J A, Chung Y J, Lee Y S. Intracellular Ca2+ mediates lipoxygenase-induced proliferation of U-373 MG human astrocytoma cells. Arch Pharm Res 1998; 21:664-670.
15. Brocchieri A, Saporiti A, Moroni M, Porta C, Tua A, Grignani G. Verapamil inhibits to different extents agonist-induced Ca2+ transients in human tumor cells and in vitro tumor cell growth. Invasion Metastasis 1996; 16:56-64
16. Malhi H, Irani A N, Rajvanshi P, Suadicani S O, Spray D C, McDonald T V, Gupta S. KATP channels regulate mitogenically induced proliferation in primary rat hepatocytes and human liver cell lines. Implications for liver growth control and potential therapeutic targeting. J Biol Chem 2000; 275:26050-26057.
17. Nilius B, Wohlrab W. Potassium channels and regulation of proliferation of human melanoma cells. J Physiol 1992; 445:537-548.
18. Rouzaire-Dubois B, Dubois J M. A quantitative analysis of the role of K+ channels in mitogenesis of neuroblastoma cells. Cell Signal 1991; 3:333-339.
19. Rouzaire-Dubois B, Dubois J M. K+ channel block-induced mammalian neuroblastoma cell swelling: a possible mechanism to influence proliferation. J Physiol 1998; 510:93-102.
20. Xu D, Wang L, Dai W, Lu L. A requirement for K+-channel activity in growth factor-mediated extracellular signal-regulated kinase activation in human myeloblastic leukemia ML-1 cells. Blood 1999; 94:139-145.
21. Wang H, Zhang Y, Cao L, Han H, Wang J, Yang B, Nattel S, Wang Z. HERG K+ channel: A regulator of tumor cell apoptosis and proliferation. Cancer Res 2002; 62:4843-4848.
22. Abdul M, Santo A, Hoosein N. Activity of potassium channel-blockers in breast cancer. Anticancer Res 2003; 23:3347-3351.
23. Ouadid-Ahidouch H, Chaussade F, Roudbaraki M, Slomianny C, Dewailly E, Delcourt P, Prevarskaya N. Kv1.1 K+ channels identification in human breast carcinoma cells: involvement in cell proliferation. Biochem Biophys Res Commun 2000; 278:272-277.
24. Ouadid-Ahidouch H, Le Bourhis X, Roudbaraki M, Toillon R A, Delcourt P, Prevarskaya N. Changes in the K+current-density of MCF-7 cells during progression through the cell cycle: possible involvement of a h-ether-a-gogo K+ channel. Receptors Channels 2001; 7:345-356.
25. Pardo L A, del Camino D, Sanchez A, Alves F, Bruggemann A, Beckh S, Stuhmer W. Oncogenic potential of EAG K+ channels. EMBO J 1999; 18:5540-5547.
26. Bauer C K, Schwarz J R. Physiology of EAG K+ channels. J Membr Biol 2001; 182:1-15.
27. Pardo L A, Bruggemann A, Camacho J, Stuhmer W. Cell cycle-related changes in the conducting properties of r-eag K+ channels. J Cell Biol 1998; 143:767-775.
28. Haskell S G. Selective estrogen receptor modulators. South Med J 2003; 96:469-476.
29. Thomas D, Gut B, Karsai S, Wimmer A B, Wu K, Wendt-Nordahl G, Zhang W, Kathofer S, Schoels W, Katus H A, Kiehn J, Karle C A. Inhibition of cloned HERG potassium channels by the antiestrogen tamoxifen. Naunyn Schmiedebergs Arch Pharmacol 2003; 368; 4148.
1. 30. Jordan V C. Chemoprevention with antiestrogens: the beginning of the end for breast cancer. Daniel G. Miller Lecture. Ann N Y Acad Sci 2001; 952:60-72.

1.-15. (canceled)

16. A method for reducing tumour growth, said method comprising the administration of a therapeutically effective amount of an ERG channel inhibitor to a subject.

17. The method as defined in claim 16, wherein said ERG channel inhibitor is an HERG channel inhibitor selected from the group consisting of: dofetilide, and cisapride.

18. The method as defined in claim 17, wherein said HERG channel inhibitor is administered intra-tumorally.

19. The method as defined in claim 18, wherein said HERG channel inhibitor is administered in an amount of from about 1 ng to about 1 g.

20. The method as defined in claim 19, wherein said HERG channel inhibitor is administered in an amount of from about 1 μg to about 100 μg.

21. The method as defined in claim 18, wherein said HERG channel inhibitor is administered in an amount of from about 0.1 ng/tumor mm³ to about 10 μg/tumor mm³.

22. The method as defined in claim 21, wherein said HERG channel inhibitor is administered in an amount of from about 10 ng/tumor mm³ to about 200 ng/tumor mm³.

23. The method as defined in claim 16, wherein said subject is a non-human mammal.

24. The method as defined in claim 16, wherein said subject is a human.

25. The method as defined in claim 16, wherein said ERG channel inhibitor is an interference RNA that downregulates the expression of an HERG channel.

26. The method as defined in claim 25, wherein said interference RNA comprises an siRNA having a sense strand comprising a portion whose target sequence is at least 90% homologous to a sequence selected from SEQ ID NOs: 1, 3 and 4 over at least 15 continuous nucleotides.

27. The method as defined in claim 26, wherein said interference RNA comprises an siRNA having a sense strand comprising a portion whose target sequence 100% homologous to a sequence selected from any of SEQ ID NOs: 1, 3 and 4.

28. The method as defined in claim 27, wherein said siRNA is dissolved in a pharmaceutically acceptable carrier.

29. A method for treating cancer in a human, said method comprising the administration of a therapeutically effective amount of a HERG channel inhibitor.

30. The method as defined in claim 29, wherein said HERG channel inhibitor is administered intra-tumorally and selected from the group consisting of: dofetilide and cisapride.

31. (canceled)

32. The method as defined in claim 30, wherein said HERG channel inhibitor is administered in an amount of from about 1 μg to about 100 μg.

33. (canceled)

34. The method as defined in claim 30, wherein said HERG channel inhibitor is administered in an amount of from about 10 ng/tumor mm³ to about 200 ng/tumor mm³.

35. A method for lowering the progression of cancerous cell proliferation, said method comprising administering to a subject in need thereof a therapeutically effective amount of a ERG channel inhibitor selected from the group consisting of: dofetilide and cisapride.

36-50. (canceled)

51. A method of inhibiting breast cancer cell growth, comprising inhibiting HERG activity in the breast cancer cells.

52. The method as defined in claim 51, wherein HERG activity is inhibited by contacting the breast cancer cells with an inhibitor of HERG activity.

53-54. (canceled)

55. A method of inhibiting a HERG channel in a cancer patient, comprising administering an effective amount of an anti-arrhythmic agent to a patient in need thereof.

56. A method of inhibiting a HERG channel in a cancer patient, comprising administering an effective amount of an esophageal sphincter contracting agent to a patient in need thereof.

57-59. (canceled)

60. The method as defined in claim 16, wherein a tumor whose growth is inhibited includes cancer cells from a cell line selected from breast cancel cell line SK-BR-3, breast cancer cell line MCF-7, breast cancer cell line BT-132, neuroblastoma cell line SHSY5Y, and murin atrial tumor cell line HL1.

61. The method of claim 29, wherein the cancer includes cancer cells from a cell line selected from breast cancel cell line SK-BR-3, breast cancer cell line MCF-7, breast cancer cell line BT-132, neuroblastoma cell line SHSY5Y, and murin atrial tumor cell line HL1.

62. (canceled)

63. The method as defined in claim 16, wherein said ERG channel inhibitor includes dofetilide.

64. The method as defined in claim 16, wherein said ERG channel inhibitor includes cisapride.
